(19) United States
(12) Patent Application Publication
(10) Pub. No.: US 2026/0295204 A1
(43) Pub. Date: Oct. 1, 2026

(54) SENSORY-RESPONSIVE WEARABLE SYSTEM WITH BIOMETRIC-CONTROLLED ADAPTIVE ACTUATION

(71) Applicant: Samantha Shael Marston, Kinnelon, NJ (US)

(72) Inventor: Samantha Shael Marston, Kinnelon, NJ (US)

(21) Appl. No.: 19/411,034

(22) Filed: Dec. 5, 2025

Related U.S. Application Data
(60) Provisional application No. 63/780,670, filed on Mar. 31, 2025.

(51) Int. Cl. A61M 21/02; A41D 1/04; A61H 9/00; A61M 21/00

(52) U.S. Cl. CPC A61M 21/02; A41D 1/04; A61H 9/0092; A61H 2201/1246; A61H 2201/165; A61H 2201/5056; A61H 2201/5097; A61H 2230/065; A61M 2021/0022; A61M 2205/3584; A61M 2205/8206; A61M 2230/06

(57) ABSTRACT

A sensory-responsive wearable system is provided for delivering adaptive compression based on biometric feedback. The system includes a wearable textile structure incorporating one or more pneumatic or otherwise adaptive actuators configured to apply variable pressure to the wearer. The wearable integrates at least one biometric sensor configured to monitor a physiological parameter indicative of changes in the wearer's physical state. A control module interprets biometric data and selectively activates an actuation assembly that inflates or deflates an expandable chamber to modulate compression. In certain embodiments, the system communicates wirelessly with an external interface for monitoring, calibration, or manual adjustment. The wearable thereby provides dynamic, real-time tactile regulation to support comfort, focus, and sensory management for neurodivergent or users who may benefit from automated pressure-based feedback.

### FIELD OF THE DISCLOSURE

The present disclosure relates generally to wearable technology and adaptive textile systems, and more particularly to wearable articles that incorporate biometric sensing, control electronics, and pneumatic or otherwise adaptive actuation mechanisms configured to provide variable compression or tactile feedback in response to changes in a wearer's physiological state, including embodiments employing soft-robotic structures integrated into garments or other wearable forms for use in smart textiles, assistive wearables, sensory-responsive apparel, and biofeedback-driven actuation technologies.

### BACKGROUND OF THE RELATED ART

Wearable articles designed to improve comfort or provide sensory support are known in the art. Conventional sensory garments typically rely on passive features such as fabric selection, seamless construction, or fixed compression levels that remain constant regardless of a wearer's changing physical state. Such passive designs do not dynamically adapt to fluctuations in physiological signals or provide responsive tactile modulation.

Biometric monitoring devices, including heart rate sensors and other physiological trackers, are also known and commonly used in consumer wearables. However, these devices generally function only as measurement tools and do not directly interface with actuators capable of delivering physical feedback or compression to the wearer. As a result, known biometric systems lack integrated mechanisms for providing real-time, tactile responses based on detected physiological changes.

Soft-robotic and pneumatic actuation technologies have been employed in applications such as prosthetics, assistive robotics, and research platforms. While these systems can produce controlled motion or pressure, they are typically not configured for integration into garments or flexible textiles suitable for continuous wear. Existing solutions often involve bulky hardware, rigid components, or configurations unsuitable for embedding within apparel.

Accordingly, there remains a need in the art for a wearable article capable of interpreting biometric information and automatically modulating applied pressure through lightweight, textile-integrated actuation mechanisms. Such a system would provide responsive, adaptive tactile feedback to support user comfort, sensory regulation, or related use cases, while maintaining the flexibility, mobility, and aesthetics expected of wearable apparel.

### DESCRIPTION OF RELATED ART

In a published U.S. patent application, US2024/0342046A1, Arnold Tobin discloses a wearable pneumatic compression apparatus. The reference describes a garment configured to deliver therapeutic compression using pneumatic chambers. However, the device provides pre-set or clinician-determined compression levels and does not integrate biometric sensing or a closed-loop control architecture capable of automatically adjusting pressure based on real-time physiological changes detected from the wearer.

In U.S. Patent Application No. US2025/0057716 A1, Divyakshi Kaushik et al. disclose a portable, daily-wear smart compression device. The system provides configurable compression for orthopedic or circulatory therapy. However, while the reference discusses wearable compression hardware, it does not teach or suggest a garment incorporating embedded biometric sensors or control logic that autonomously modulates compression based on physiological feedback, nor does it disclose textile-integrated soft or pneumatic actuation systems designed for continuous sensory regulation.

In a further U.S. application, US2025/0275716 A1, Joseph J. et al. describe a sensorized wearable garment that integrates physiological sensors for monitoring user metrics. Although the sensors may be embedded in the fabric, the reference is limited to data collection and fails to provide any mechanism for delivering mechanical pressure or tactile feedback to the wearer. Accordingly, the application does not teach adaptive actuation or pneumatic modulation driven by biometric data.

U.S. Pre-Grant Publication No. US2017/0100300 A1, “Advanced Compression Garments and Systems,” by Scott Rapp et al., discloses garments incorporating sensors that measure compression levels exerted on a user. While the system can monitor applied pressure, it does not disclose textile-integrated actuators or any capability for dynamically adjusting compression in response to physiological conditions, nor does it describe a processor-driven feedback loop based on biometric inputs.

Another published application, US2020/0093679 A1, by Harshal Arun Sonar et al., describes a soft, portable, wearable pneumatic interactive suit using inflatable actuators for haptic or assistive movement. Although the reference teaches pneumatic actuation, the system relies on structural components and configurations unsuitable for integration into lightweight garments intended for daily wear. Moreover, it does not disclose biometric-triggered adaptive compression or any wearable architecture tailored for sensory regulation.

In yet another reference, U.S. Patent Application No. US2014/0070957 A1, Gianluigi et al. disclose a wearable communication platform in which sensors collect physiological data and provide haptic or audio feedback to the wearer. While the platform demonstrates physiological sensing and feedback delivery, it does not teach the use of pneumatic or soft-robotic actuators to apply adjustable mechanical compression, nor does it disclose a closed-loop system in which biometric data directly controls garment-integrated actuators.

None of the above-discussed prior art teaches or suggests a fully wearable, textile-integrated system that combines biometric sensing, processor-controlled pneumatic or adaptive actuation, and dynamic pressure modulation for real-time sensory regulation. The existing art lacks a garment or wearable system capable of automatically interpreting physiological signals and providing responsive tactile compression through embedded actuators in a lightweight, flexible textile form factor suitable for continuous daily wear.

### SUMMARY OF THE INVENTION

The present disclosure provides a sensory-responsive wearable system configured to deliver adaptive compression or tactile pressure to a wearer based on biometric feedback. The system generally includes a wearable textile assembly, a biometric sensing module, a control module, and an actuation assembly capable of producing variable compression in response to detected physiological conditions. The system is designed to operate as a lightweight, fully wearable platform that continuously monitors biometric inputs and adjusts applied pressure in real time.

In one aspect, the wearable textile assembly comprises a garment or other wearable article formed from flexible materials that permit integration of sensors, actuators, tubing, and conductors without compromising comfort or mobility. The textile structure may include internal channels, seams, or fabric layers configured to house pneumatic chambers, inflatable bladders, soft-robotic actuators, or other adaptive actuation mechanisms. The textile assembly may further include laser-cut openings, patterned slits, or reinforced pathways that enable controlled expansion or contraction of embedded actuators.

The biometric sensing module includes one or more sensors configured to detect physiological parameters such as heart rate, pulse waveform characteristics, or other signals indicative of changes in the wearer's physical state. The sensor may be positioned directly against the skin, embedded within a lining layer, or disposed along an interior region of the garment. The module may generate analog or digital outputs corresponding to real-time physiological activity.

The control module includes one or more processors, memory components, and signal-processing circuitry configured to acquire biometric data, filter or interpret the data, and determine whether an actuation event is required. In certain embodiments, the control module implements threshold-based logic, in which actuator activation is triggered when biometric readings exceed or fall below predefined values. In other embodiments, the control module may execute algorithms that evaluate trends, amplitude variations, or temporal patterns in biometric measurements.

The actuation assembly comprises one or more pneumatic, soft-robotic, or other adaptive actuation mechanisms configured to apply pressure to the wearer. In some embodiments, the assembly includes inflatable chambers positioned within the textile structure. These chambers may be selectively inflated or deflated using compact pumps, valves, or airflow-control components. Activation of the actuation assembly results in localized or distributed pressure being applied to targeted regions of the wearer's body.

The pumps utilized in the actuation assembly may be miniature electric pumps capable of generating positive or negative pressure. A first pump may be designated for inflation, while a second pump may be designated for deflation. A valve or manifold system may direct airflow between pumps and the pneumatic chamber, allowing fine control over compression intensity, speed of actuation, and overall pressure regulation.

The control module may be electrically coupled to the actuation assembly through one or more motor drivers or power-management circuits. These circuits regulate actuator power levels, control pump directionality, and prevent over-pressurization. In one embodiment, the control module communicates with a motor driver that adjusts pump voltage or duty cycle in accordance with the required actuation force.

The system may include a power supply such as a battery pack, energy cell, or rechargeable module. The power supply may be integrated into the garment at a location chosen to minimize interference with wearer movement. Power management circuitry may monitor battery health, distribute voltage to system components, and ensure consistent performance during continuous operation.

In certain embodiments, the control module includes wireless communication capabilities, enabling the wearable system to interface with external devices such as smartphones, tablets, or cloud-based platforms. Wireless communication may allow a user or caregiver to modify threshold settings, monitor biometric readings, or manually override actuator behavior.

The system may operate in a fully autonomous closed-loop configuration, continuously collecting biometric data, interpreting the data, and activating or deactivating the actuation assembly without user intervention. Responsive adjustments may occur in real time, ensuring that applied pressure aligns with the wearer's physiological state at any given moment.

In some embodiments, the algorithm executed by the control module includes filtering routines for eliminating noise from biometric signals, ensuring reliable detection of physiological changes. Thresholds for actuation may be preset, learned over time, or dynamically adapted based on historical data.

The wearable system may support multiple actuation zones, with independent pneumatic or soft-robotic modules positioned in different regions of the garment. Each module may be separately controlled, allowing localized compression patterns tailored to specific wearer needs or sensory preferences.

The garment structure may incorporate modular components, enabling individual actuators, tubes, sensors, or electronic modules to be replaced, serviced, or upgraded without removing the entire garment from use. This modularity supports both prototyping and long-term usability.

In certain embodiments, the textile assembly may use composite fabrics, hybrid materials, or layered structures to optimize flexibility, breathability, and actuator performance. For example, outer layers may provide structural support, while inner layers may facilitate actuator expansion or maintain contact between the biometric sensor and the wearer's skin.

The actuation assembly may also include pressure-relief mechanisms, compliance materials, or structural reinforcements that ensure uniform pressure distribution and prevent excessive localized force. Such features improve wearer comfort and reduce the risk of mechanical fatigue in the actuators.

The system may incorporate environmental or contextual sensing in addition to biometric sensing. For example, ambient temperature, user movement, or posture may be measured to refine actuation responses. Although such sensors are optional, they may enhance the accuracy and responsiveness of compression control.

The wearable system may include feedback indicators such as onboard LEDs, haptic cues, or digital notifications through a connected device. These indicators may confirm system operation, alert the user to threshold conditions, or signal activation events.

The control module may use preconfigured modes of operation, such as a “calming mode,” “compression mode,” “relief mode,” or “adaptive mode,” each employing different threshold values or actuator behaviors. These modes may be selected manually or automatically based on wearer needs.

The garment may be configured to operate quietly and discreetly, reducing noise generated by pneumatic pumps or valves. Sound-dampening structures or slow-actuation routines may be used to minimize auditory distractions.

The system is designed to be lightweight and body-conforming, enabling use during everyday activities without restricting movement or causing discomfort. The integration of components into the textile structure ensures a natural appearance and minimizes bulk.

In certain embodiments, washable or detachable electronic modules may be employed, permitting the garment to be laundered without damaging sensors or actuators. Waterproof or water-resistant housings may be used for additional protection.

The disclosed wearable system provides improved sensory regulation, user comfort, and adaptability compared to traditional static compression garments. By combining biometric sensing with real-time actuation, the system offers a dynamic method for supporting user focus, stress management, and overall well-being.

The present invention therefore addresses longstanding deficiencies in compression garments, biometric monitoring devices, and soft-robotic systems by providing a unified, textile-integrated, responsive platform capable of interpreting physiological cues and delivering adaptive tactile modulation in real time.

### BRIEF DESCRIPTION OF THE DRAWINGS

The accompanying drawings illustrate several embodiments of the invention and, together with the description, serve to explain the principles of the invention according to the embodiments. One skilled in the art will recognize that the particular embodiments illustrated in the drawings are merely exemplary, and are not intended to limit the scope of the present invention.

FIG. 1 is a block diagram illustrating an electronic and pneumatic control system, according to some embodiments of the present disclosure.

FIG. 2 is a block diagram illustrating multiple views of an electronic circuit assembly, according to some embodiments of the present disclosure.

FIG. 3 is a diagram illustrating front and rear views of a wearable garment incorporating soft-robotic actuation elements, according to some embodiments of the present disclosure.

FIG. 4 is a flowchart illustrating control logic for operating a sensory-responsive wearable system, according to some embodiments of the present disclosure.

### DETAILED DESCRIPTION OF THE INVENTION

Unless otherwise defined, all technical terms used herein related to wearable technology, soft robotics, pneumatic actuation, biometric sensing, embedded electronics, microcontroller systems, and physiological signal processing have the same meaning as commonly understood by one of ordinary skill in the relevant arts of wearable systems, robotics, biomedical instrumentation, and textile-integrated electronics. It will be further understood that terms such as “pneumatic actuator,” “soft-robotic muscle,” “biometric sensor,” “microcontroller,” “physiological signal,” and other domain-specific terms used throughout this disclosure should be interpreted as having meanings consistent with their usage in the context of this specification and the current state of smart-garment and soft-robotics technology. These terms should not be interpreted in an idealized or overly formal sense unless expressly defined herein. For clarity and conciseness, well-known functions, circuit constructions, pneumatic arrangements, or embedded-system principles may not be described in detail.

The terminology used herein describes particular embodiments of the sensory-responsive wearable system and is not intended to be limiting. As used herein, singular forms such as “a biometric sensor,” “a soft-robotic actuator,” “a pneumatic pump,” “a microcontroller,” and “a garment-integrated control module” are intended to include plural forms as well, unless the context clearly indicates otherwise. Similarly, references to “physiological input,” “pneumatic actuation,” “compression adjustment,” or “control logic” should be understood to include multiple instances, components, cycles, or operating modes where applicable.

With reference to the use of the words “comprise,” “comprises,” or “comprising” in describing the components, elements, or functionalities of the embodiments disclosed herein, and in the following claims, unless the context requires otherwise, these terms are intended to be interpreted inclusively rather than exclusively. For example, the phrase “a system comprising a biometric sensor and a pneumatic actuator” should be understood to mean including but not limited to the described elements, and may include additional sensors, actuators, processors, valves, pumps, or communication components not expressly identified. Each instance of these terms is to be interpreted inclusively in construing both the description and the claims, particularly given the modular and adaptable nature of wearable robotic systems and textile-embedded electronics.

Furthermore, terms such as “connected,” “coupled,” “in communication with,” or “operatively linked,” as used in describing the interactions among sensors, microcontrollers, pumps, valves, garments, and electronic assemblies, should be interpreted to include both direct physical or electrical connections and indirect connections through one or more intermediary components unless explicitly stated otherwise. References to “processing,” “analyzing,” “determining,” or “controlling” should be understood to encompass real-time, near-real-time, periodic, event-driven, or batch-mode operations unless specifically limited to a particular processing modality within the context.

According to some embodiments of the present disclosure, the sensory-responsive wearable system generally includes a biometric sensing module, a control module, and a pneumatic or soft-robotic actuation assembly integrated into a textile garment. The system is configured to monitor a physiological characteristic of a wearer, interpret the biometric data, and selectively actuate an inflatable or contractile element to provide adaptive compression or tactile stimulation.

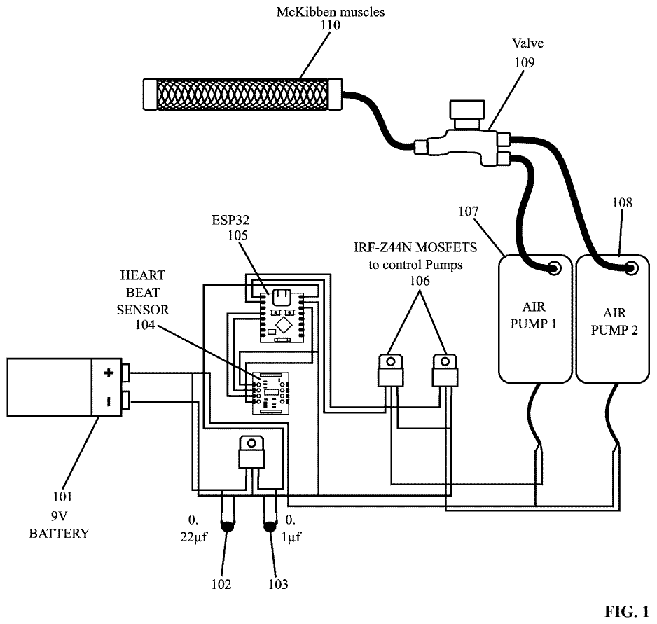

FIG. 1 illustrates an electronic and pneumatic control system used to operate a McKibben artificial muscle 110 or other soft-robotic actuator. As shown, the system may include a 9V power source 101, a pulse sensor, an ESP32 microcontroller 105, one or more miniature air pumps 107 and 108, a solenoid or directional control valve 109, MOSFET driver components configured to operate the pumps 106, airflow channels, and associated circuitry configured to receive biometric data and modulate pneumatic pressure accordingly.

In some embodiments, the biometric sensing module includes a pulse sensor configured to detect heart-rate data from the wearer. The pulse sensor may operate using photoplethysmography (PPG), electrical conduction measurement, or other suitable sensing mechanisms. The sensor may output an analog or digital signal representing the wearer's pulse waveform, which may be filtered or conditioned prior to processing by the ESP32 microcontroller 105.

The sensor may be embedded within a portion of the garment designed to maintain skin contact, or mounted on a small auxiliary strap or band. In some embodiments, the sensor is communicatively coupled to the control module through wired conductors integrated into the textile layers of the garment. Wireless sensor embodiments may also be employed, and the resulting physiological data may be used by the microcontroller 105 to drive activation of the air pumps 107, 108 and valve 109 to control inflation and deflation of the McKibben muscle actuator 110.

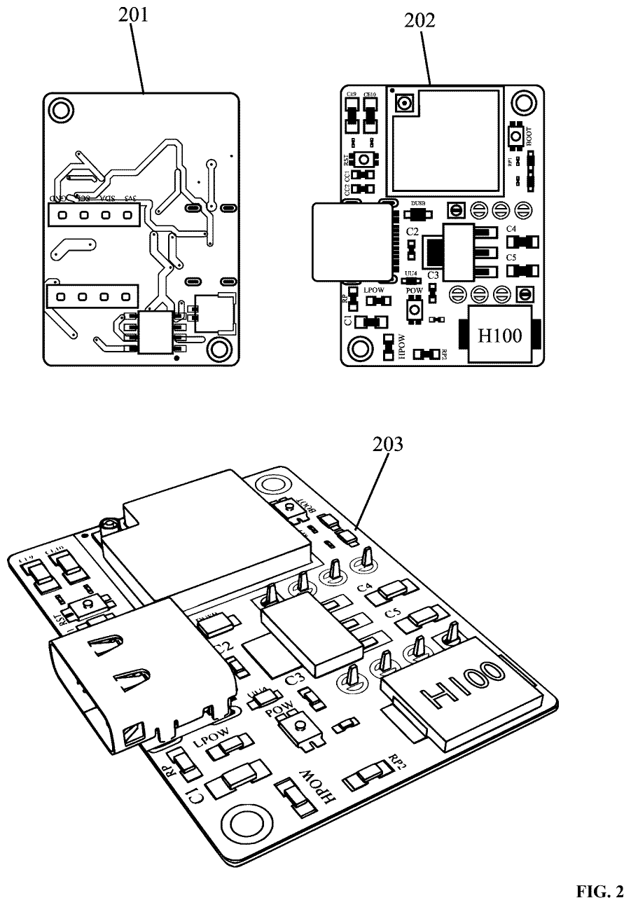

FIG. 2 illustrates multiple views 201, 202, 203 of an electronic circuit assembly forming the control module of the wearable system. According to some embodiments, the control module includes a microcontroller, such as an ESP32 device, configured to execute programmed logic that evaluates incoming biometric signals and determines whether an actuation condition has been met.

The PCB may include signal-conditioning circuits, motor-driver components, wireless communication elements, voltage regulators, and connectors for interfacing with sensors, pumps, and valves. The PCB may be arranged in a compact footprint to support wearable applications and may be enclosed within a protective housing attached to the garment.

In some embodiments, wireless communication functionality, when present, may enable connection to an external interface, such as a smartphone or IoT platform. In some embodiments, the system may display real-time heart-rate readings, actuator status, or control parameters through an application.

As shown in FIG. 1, the actuation assembly may include a McKibben artificial muscle 110, an inflatable chamber, a soft-robotic actuator, or any other structure configured to apply pressure in response to pneumatic input. The actuator may be integrated into the garment through fabric channels, laser-cut slits, or layered textile constructions.

According to some embodiments, the pneumatic assembly includes two miniature pumps 107,108 one for inflation and one for deflation and a solenoid or directional valve controlling airflow 109. Tubing may connect the pumps and valve to the actuator. The pumps may operate under the control of the microcontroller, 106 to increase or decrease internal air pressure within the actuator.

The actuator may expand, contract, or otherwise deform when pneumatic pressure is applied. In McKibben muscle embodiments, the braided structure shortens under internal pressurization, providing a contractile force.

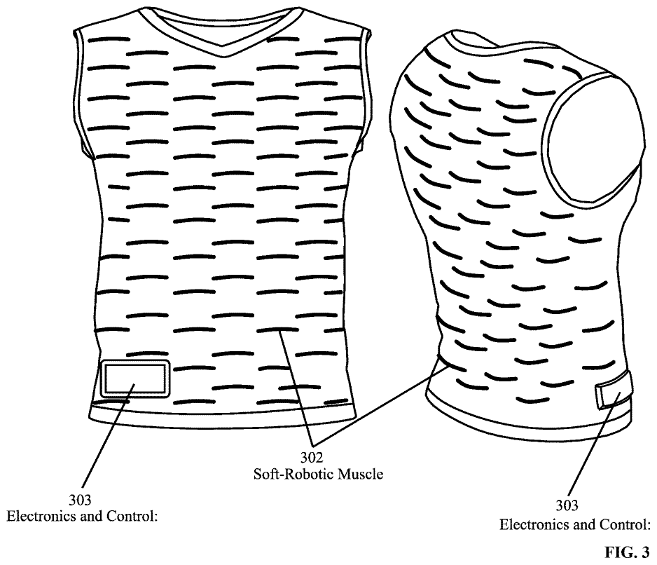

FIG. 3 illustrates front and rear views of a garment incorporating the actuation assembly. According to some embodiments, the garment includes textile layers configured to house soft-robotic muscle elements 302, actuator components, tubing, and wiring. The textile structure may include laser-cut openings, patterned slits, or a flexible weave pattern that accommodates expansion and contraction of the soft-robotic actuator during use.

The garment may further include an electronics and control module 303 integrated into a lower or side region of the garment. The control module may contain power electronics, the microcontroller, driver circuitry, and communication hardware. The garment body may be fabricated from silk, synthetic fibers, elastic materials, or composite fabrics, depending on the desired mechanical and aesthetic characteristics. Reinforced seams or interior channels may be employed to maintain actuator alignment and prevent unwanted shifting during wear.

In some embodiments, the garment may be designed to fit tightly or loosely depending on the intended application. For embodiments requiring deep-pressure stimulation or compression-based regulation, the garment may include form-fitting regions configured to maximize transfer of actuation forces from the soft-robotic muscles 302 to the wearer. The electronics and control module 303 may be positioned or distributed to preserve comfort while maintaining stable electrical connections to the actuator assembly.

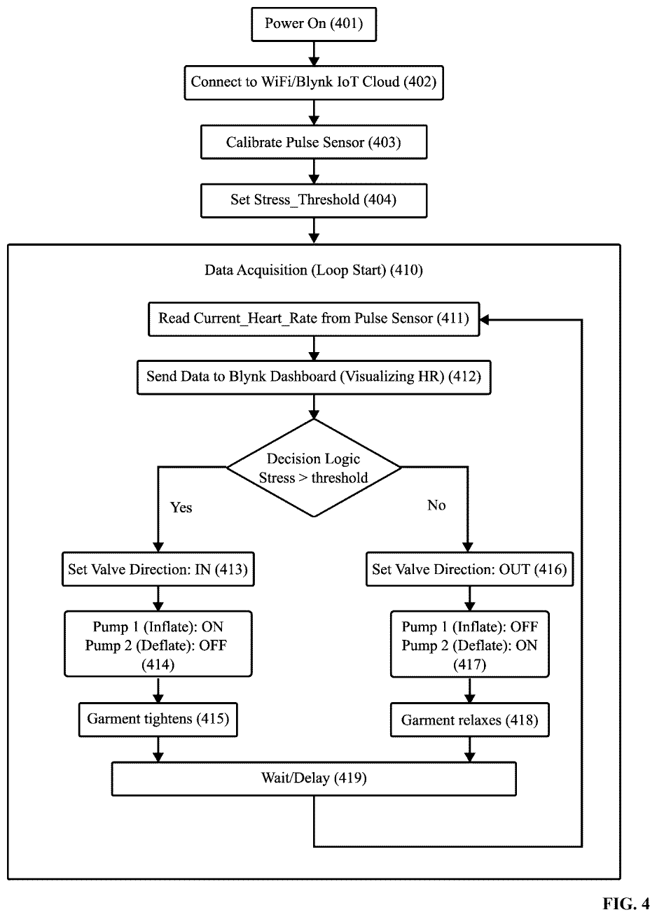

FIG. 4 is a flowchart illustrating an exemplary control process executed by the control module. According to some embodiments, the process begins at a power-on stage 401, after which the system establishes a connection to a WiFi or Blynk IoT cloud interface 402. The pulse sensor is then calibrated 403, and a stress threshold is set 404. The system thereafter enters a data-acquisition loop 410, during which the microcontroller reads the current heart-rate value from the pulse sensor 411 and transmits the data to a remote dashboard or visualization interface 412.

The microcontroller may then execute a decision-logic routine configured to determine whether the measured stress or heart-rate level exceeds the predefined threshold. If the condition is satisfied, the system may set the valve direction to an inflow configuration 413 and activate Pump 1 to inflate the actuator while deactivating Pump 2 414. In this mode, the garment may tighten 415, providing deep-pressure stimulation or other calming effects as desired for sensory regulation. The system may then execute a timing delay 419 before acquiring the next reading.

If the measured stress or heart-rate level does not exceed the threshold, the microcontroller may configure the valve to an outflow direction 416 and deactivate Pump 1 while activating Pump 2 to deflate the actuator 417. In this configuration, the garment may relax 418 to reduce applied pressure. A timing delay 419 may again be imposed before returning to the next sampling cycle.

The system may operate in a closed-loop cycle that repeatedly samples biometric data and adjusts actuator pressure accordingly. The logic may further incorporate filtering routines, smoothing algorithms, averaging windows, debounce logic, or artifact-reduction techniques to improve fidelity of physiological measurements and ensure stable actuation control.

In some embodiments, user-adjustable parameters such as stress-threshold values, actuator intensity levels, response timing, or delay intervals may be configured through an external interface, including but not limited to mobile applications, IoT dashboards, or wireless controllers.

Although many embodiments utilize pneumatic actuation as shown in the inflation 414 and deflation 417 sequences, the disclosed system is not limited to air-driven mechanisms. Other reversible actuation technologies may be used in addition to, or in place of, pneumatic components, including hydraulic systems, electroactive polymers, shape-memory alloys, dielectric elastomers, or other adaptive materials.

Similarly, in certain embodiments while heart-rate sensing 411 is illustrated, the system may utilize any biometric or contextual signal. Examples include respiratory rate, galvanic skin response, blood-oxygen saturation, motion or posture data, environmental cues, or multi-sensor fusion inputs, any of which may be integrated into the decision logic to trigger the adaptive actuation process.

In some embodiments, the garment incorporating this control logic may take multiple forms, including vests, tops, belts, sleeves, undergarments, or accessory structures. The placement of the actuators, pumps, valve, and control modules may vary depending on ergonomic requirements, intended therapeutic effects, or aesthetic considerations.

The control module implementing the sequence of operations 401-419 may be embodied using microcontrollers such as the ESP32, application-specific integrated circuits (ASICs), distributed processing networks, or hybrid architectures. Wireless communication, battery-management systems, and PCB-level integration may also be incorporated to support reliable operation.

Additional embodiments may include machine-learning-based adaptive thresholding, multi-zone compression control, or integration with external assistive-technology ecosystems.

The embodiments described herein provide a sensory-responsive wearable system that integrates biometric sensing, microcontroller-based analysis, and soft-robotic or pneumatic actuation to deliver adaptive compression and tactile modulation in real time, offering a meaningful approach to enhancing user comfort, supporting emotional regulation, improving sensory processing, and advancing inclusive assistive-technology solutions. The combination of textile-integrated actuators, embedded electronics, and closed-loop physiological control represents a significant development in wearable robotics and smart-garment technology. Although specific embodiments have been presented to illustrate the structure and operation of the system, numerous modifications, substitutions, and variations may be made without departing from the intended scope of the invention, including alterations in actuator types, biometric sensors, garment materials, control logic, and communication interfaces, and all such alternatives are considered to fall within the spirit and scope of the present disclosure as supported by the appended claims and their full range of equivalents.

What is claimed is:

1. A sensory-responsive wearable system comprising:
a biometric sensor configured to detect a physiological signal of a wearer;
a microcontroller in communication with the biometric sensor and configured to process the physiological signal;
a pneumatic actuation assembly including at least one pump, at least one valve, and at least one inflatable or contractile actuator;
a control module operatively coupled to the microcontroller and the pneumatic actuation assembly; and
a garment configured to house the actuator and at least a portion of the control module,
wherein the microcontroller is configured to activate the pneumatic actuation assembly to adjust pressure applied by the actuator in response to the processed physiological signal.

2. The system of claim 1, wherein the physiological signal comprises heart-rate data.

3. The system of claim 1, wherein the microcontroller comprises an ESP32 processor.

4. The system of claim 1, wherein the actuator comprises a McKibben artificial muscle.

5. The system of claim 1, wherein the pneumatic actuation assembly comprises a first pump configured to inflate the actuator and a second pump configured to deflate the actuator.

6. The system of claim 1, wherein the microcontroller is configured to compare the physiological signal to a threshold and activate the actuator when the threshold is exceeded.

7. The system of claim 1, wherein the garment includes textile channels, laser-cut slits, or fabric pathways configured to receive the actuator.

8. The system of claim 1, further comprising a printed circuit board supporting the microcontroller, driver circuitry, and power-management components.

9. The system of claim 1, wherein the control module is configured to communicate with a remote interface through a wireless communication protocol.

10. The system of claim 1, wherein the pneumatic actuation assembly comprises at least one pump configured to generate airflow, at least one valve configured to direct or regulate airflow, and at least one inflatable or contractile actuator configured to apply pressure to the wearer.

11. The system of claim 1, wherein the microcontroller is configured to execute instructions stored on a non-transitory computer-readable medium, the instructions causing the microcontroller to analyze physiological data, determine an actuation condition, and control the at least one pump and the at least one valve to modulate compression applied to the wearer.

12. A wearable garment comprising:
a textile structure having a front portion and a rear portion;
at least one soft-robotic actuator integrated into or disposed upon the textile structure;
a control module comprising a microcontroller, driver circuitry, and at least one pneumatic pump; and
a biometric sensor configured to provide physiological data to the control module,
wherein the control module is configured to inflate or deflate the at least one soft-robotic actuator to provide adaptive compression based on the physiological data.

13. The garment of claim 12, wherein the at least one soft-robotic actuator extends across both the front and rear portions of the textile structure.

14. The garment of claim 12, wherein the textile structure comprises silk, elastic fibers, synthetic materials, or composite fabrics.

15. A method for providing adaptive compression using a wearable actuation system, the method comprising:
receiving, by a biometric sensor, a physiological signal from a wearer;
processing, by a microcontroller, the physiological signal to determine an actuation condition;
actuating at least one pump and at least one valve to inflate or deflate an actuator integrated within a garment; and
applying, by the actuator, adaptive compression to the wearer in response to the actuation condition.

16. The method of claim 15, wherein determining the actuation condition comprises comparing the physiological signal to a predefined threshold.

17. The method of claim 15, further comprising transmitting the physiological signal to a remote monitoring or cloud-based platform.

18. The method of claim 15, further comprising generating airflow using at least one pump and directing the airflow using at least one valve to inflate or deflate an inflatable or contractile actuator integrated within the garment.

19. The method of claim 15, wherein controlling inflation or deflation of the actuator comprises executing, by the microcontroller, instructions stored on a non-transitory computer-readable medium configured to analyze physiological data, determine an actuation condition, and modulate the actuator accordingly.
